(19) United States
(12) Patent Application Publication
(10) Pub. No.: US 2026/0224809 A1
(43) Pub. Date: Aug. 6, 2026

(54) INSULIN PATCH PUMP WITH IMPROVED ACTUATION MECHANISM

(71) Applicant: Tandem Diabetes Care, Inc., San Diego, CA (US)

(72) Inventors: Giovanni Casula, San Diego, CA (US); Amin Amirouche, San Diego, CA (US); Maxime Ettori, San Diego, CA (US)

(21) Appl. No.: 19/457,955

(22) Filed: Jan. 23, 2026

Related U.S. Application Data
(60) Provisional application No. 63/752,049, filed on Jan. 31, 2025.

(51) Int. Cl. A61M 5/168; A61M 5/142

(52) U.S. Cl. CPC A61M 5/16813; A61M 5/14248

(57) ABSTRACT

In accordance with one aspect, a medication infusion device is provided that includes a patch pump configured to be removably adhered to a wearer's skin for delivering doses of medication (e.g., insulin) from a cartridge (e.g., a pre-filled cartridge) through a transcutaneous portion. The medication infusion system may include a pumping system configured to advance a plunger in the cartridge to cause medication in the cartridge to be delivered to the patient. The pumping mechanism may be an improved system that reduced errors in medication administration.

### PRIORITY CLAIM

The present application claims the benefit of U.S. Provisional Patent Application No. 63/752,049 filed January 31, 2025, which is hereby incorporated herein by reference in its entirety.

### TECHNICAL FIELD

The present disclosure relates generally to systems and methods for delivering medication such as insulin to a user, for example, wearable insulin pumps having a patch-style form factor for adhesion to a user’s body surface.

### BACKGROUND

Wearable insulin pumps are known for providing a Type I Diabetes Mellitus patient with small doses of short acting insulin continuously (basal rate). The devices also can be used to deliver variable amounts of insulin when a meal is consumed (bolus). The basal insulin rates are usually programmed in a pump by a physician, and one or multiple basal settings may be programmed in the pump based on the patient's needs. The patient may program the amount of insulin for a mealtime bolus directly on the pump. Most pumps also include bolus calculators to help the patient determine the amount of insulin the patient may need at mealtime based on the patient's glucose levels and the amount of carbohydrates the patient may consume. The objective is to control the patient's blood glucose level within a desired range. Some such insulin pumps are coupled to an adhesive patch that permits the pump to be directly adhered to a user's body surface, for example the abdomen, and are referred to as “patch pumps.” In addition, some previously known systems were configured to interface wirelessly with a continuous glucose monitor, which typically also may be disposed on a patch designed to be adhered to the user's body. Other previously known systems employ still further modules designed to monitor user activity and report that activity to a controller associated with the patch pump to titrate the insulin delivery in accordance with the user's activity level.

U.S. Pat. No. 11,806,502, the entire contents of which are incorporated herein by reference, assigned to the assignee of the instant application, describes a self-contained patch pump having a motor-actuated syringe together with a microdosing pump chamber.

U.S. Pat. No. 11,813,428, the entire contents of which are incorporated herein by reference, assigned to the assignee of the instant application, describes a drug delivery device comprising a pumping system and a liquid reservoir fluidly connected to a delivery system outlet. The liquid reservoir has an elastic plunger sealingly slidable within a container wall of the liquid reservoir for expelling liquid out of the reservoir.

U.S. Pat. No. 11,529,460, the entire contents of which are incorporated herein by reference, assigned to the assignee of the instant application, describes systems and methods for delivering medication such as insulin that are user-friendly, environmentally-friendly, lower cost, discreet, less prone to errors, and/or that deliver precise, repeatable doses of medication.

### SUMMARY

Provided herein are systems and methods for delivering medication, such as insulin, that are user-friendly, environmentally-friendly, lower cost, discreet, less prone to errors, and/or that deliver precise, repeatable doses of medication, as well as accessories for applying and managing the same. In embodiments, the system includes a wearable insulin pump having a patch-style form factor for adhesion to a user's body surface.

In accordance with one aspect, a medication infusion device is provided that includes a patch pump configured to be removably adhered to a wearer's skin for delivering doses of medication (e.g., insulin) from a cartridge (e.g., a pre-filled cartridge) through a transcutaneous portion. The medication infusion system may include a pumping system configured to advance a plunger in the cartridge to cause medication in the cartridge to be delivered to the patient. The pumping mechanism may be an improved system that reduced errors in medication administration.

In an embodiment, a medication infusion device comprises a patch pump configured to be removably adhered to a wearer’s skin for delivering doses of medication from a container through a transcutaneous portion. The patch pump can include a dosing tube configured to receive medication, a plurality of levers configured to contact the dosing tube to move a predetermined dose of medication towards the transcutaneous portion and a circular cam comprising a shaft oriented in a first plane and a circular plate oriented in a second plane. The circular plate can be coupled to the shaft and include a top surface having one or more raised surfaces extending from the top surface configured to move each of the plurality of levers from a lowered position to a raised position in a series of steps upon rotation of the shaft. Contact surfaces on the plurality of levers at which the plurality of levers are contacted by the raised surfaces can be configured to inhibit lateral movement of each the plurality of levers when the plurality of levers are moved from the lowered position to the raised position. Rotation of the shaft can deliver the predetermined dose of medication towards the wearer.

In embodiments, a medication infusion device comprises a patch pump configured to be removably adhered to a wearer’s skin for delivering doses of medication from a container through a transcutaneous portion. The patch pump can include a dosing tube configured to receive medication, a plurality of levers configured to contact the dosing tube to move a predetermined dose of medication towards the transcutaneous portion and a circular cam comprising a shaft oriented in a first plane and a circular plate oriented in a second plane. The circular plate can be coupled to the shaft and include a top surface having one or more raised surfaces extending from the top surface configured to move each of the plurality of levers from a lowered position to a raised position in a series of steps upon rotation of the shaft. Contact surfaces on the plurality of levers at which the plurality of levers are contacted by the raised surfaces can be positioned such that forces transmitted to the plurality of levers by the raised surfaces when the plurality of levers are moved from the lowered position to the raised inhibit lateral movement of each the plurality of levers. Rotation of the shaft can deliver the predetermined dose of medication towards the wearer.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an exemplary medication infusion system according to the disclosure.

FIG. 2 is a diagram showing exemplary attachment zones for the patch pump of FIG. 1 and an external sensor.

FIGS. 3A-3B depict an exemplary patch pump according to the disclosure.

FIG. 4 depicts an exploded view of the patch pump of FIGS. 3A-3B.

FIG. 5 is a schematic depiction of an exemplary pusher and microdosing system for a patch pump according to the disclosure.

FIG. 6 is an exploded view of a cap assembly for a patch pump system according to the disclosure.

FIGS. 7A and 7B depict details of a circular cam and lever system of the patch pump of FIG. 6.

FIGS. 8A-8D schematically depict operation of the cam and lever system of the patch pump of FIG. 6.

FIGS. 9A-9C schematically depict operation of an improved cam and lever system according to the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

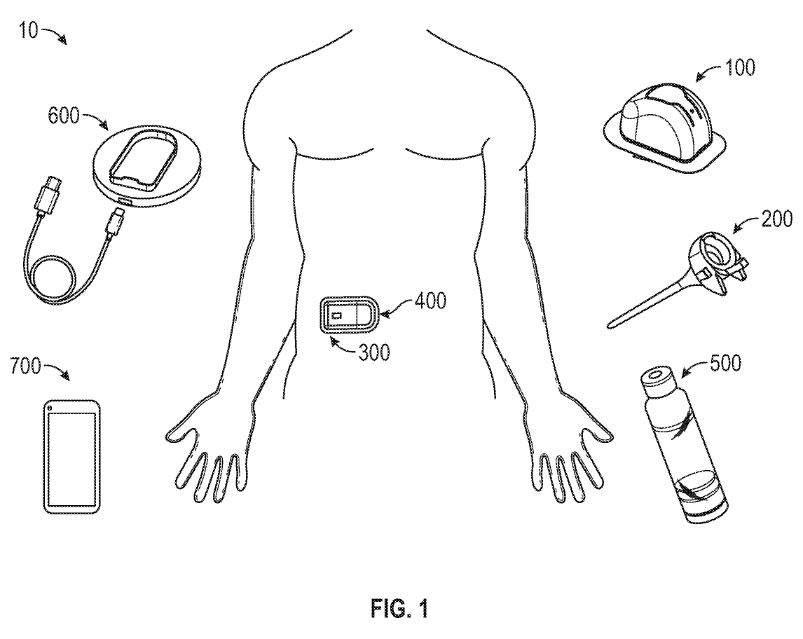

Referring to FIG. 1, an exemplary medication infusion system including a patch pump for delivering medication is described. In FIG. 1, components of the system are not depicted to scale on either a relative or absolute basis. Medication infusion system 10 can include applicator 100, cannula 200, pump 300, cap 400, cartridge 500, charging system 600 and/or software application 700. Preferably, applicator 100, cannula 200, cap 400, and cartridge 500 are disposable components that may be replaced approximately every 3-10 days and/or once the pre-filled cartridge is empty, while pump 300 is reusable and may last for an extended period of time, e.g., approximately 2-4 years. As such, pump 300 may be used with many different applicators, cannulas, caps, and pre-filled cartridges. Such a configuration is expected to promote sanitary use of the system, as the components exposed to the patient and the insulin are disposable, while reducing costs for components containing more expensive electronics, e.g., pump 300, charging system 600, and/or software application 700, which may be used repeatedly. In some embodiments, system 10 includes a second pump, such that the wearer may charge a battery of the second pump while using the first pump and vice versa. In this manner, the wearer will always have a pump that is charged and ready to be used once the cartridge of the pump in use is empty. Further, this system can be designed to reduce waste while reducing the number of times the wearer is required to insert a new cannula. Medication infusion system 10 may be used to apply cannula 200 and a pad to a wearer and to deliver medication through cannula 200 via a patch pump coupled to the pad. Further details regarding such a system can be found in U.S. Patent Publication No. 2022/0379014, which is hereby incorporated herein by reference in its entirety.

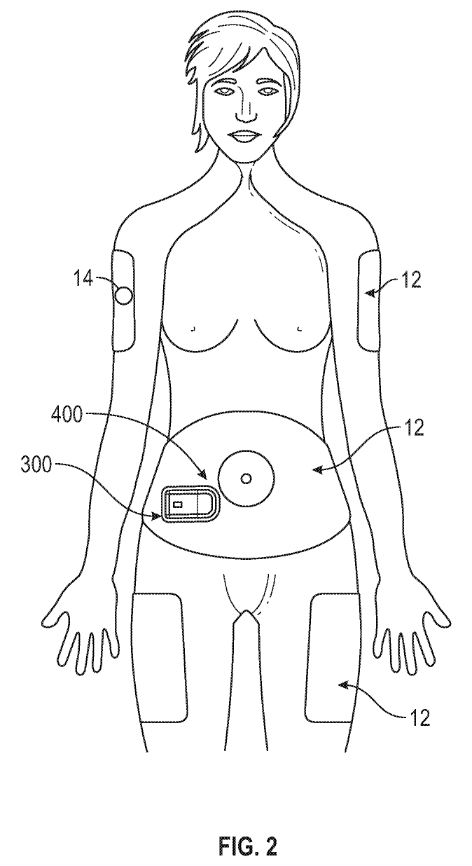

Referring now to FIG. 2, exemplary attachment zones for the patch pump and an optional external sensor, such as a continuous glucose monitoring sensor are illustrated. Attachment zones 12 illustrate several locations on the wearer's body where the applicator may attach the adhesive pad and insert the cannula and to which the patch pump is secured. For example, the patch pump may be secured to the upper arms, abdomen, or thighs of the wearer. As will also be understood by one of ordinary skill in the art, the patch pump may be secured to other locations on the wearer.

The patch pump also may be operatively coupled to an optional continuous glucose monitoring sensor, which may transmit data to a controller of the patch pump, which data may be used to adjust the time of insulin delivery or the amount of each dose. Preferably, the patch pump receives data from continuous glucose monitoring sensor 14, which is configured to be attached within attachment zones 12.

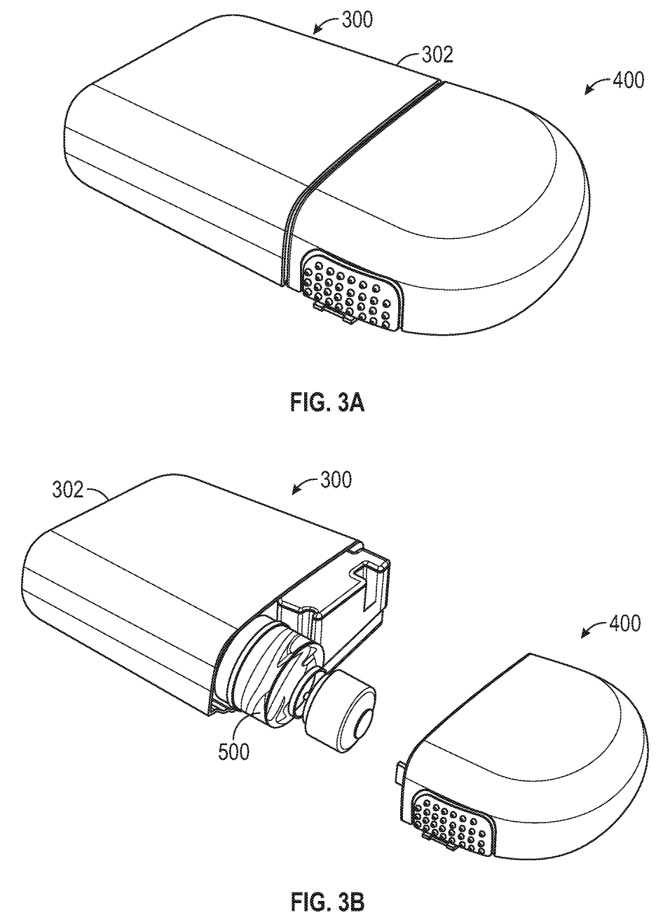

Referring now to FIGS. 3A and 3B, perspective views of an exemplary patch pump and pump-cap assembly according to the present disclosure are depicted. The patch pump is configured to attach to the adhesive pad secured to the wearer and to deliver doses of medication through the inserted cannula. The patch pump preferably includes a reusable pump, a disposable cap, a disposable pre-filled cartridge of medication, and a pad.

The patch pump may include pump 300 preferably designed to be used for an extended period of time (e.g., 2-4 years), and cap 400 preferably designed to be replaced after a much shorter period of time (e.g., 3-5 days). The patch pump also may include a pre-filled cartridge of medication, such as cartridge 500, which may be filled during manufacturing or by the wearer prior to inserting cartridge 500 into the pump. Cartridge 500 is configured to be inserted into the patch pump such that cartridge 500 is completely enclosed within the patch pump. For example, cartridge 500 may be inserted first into pump 300 such that a portion of cartridge 500 remains outside of pump housing 302. Cap 400 then may be coupled to cartridge 500 such that an inflow needle disposed within cap 400 pierces the cartridge cap of cartridge 500. While still maintaining inflow needle within cartridge 500, cap 400 then may be rotated relative to pump 300 to lock cap 400 to pump 300, thereby coupled the cap-pump assembly to the pad and the pump.

Pump 300 may include a motor disposed within pump housing 302, the motor configured to move a pusher coupled to the plunger of cartridge 500 such that insulin is advanced through an inflow needle of cap 400 and to a microdosing system designed to measure and deliver predetermined doses of medication. The same motor may simultaneously activate the plunger of the cartridge and the microdosing system, for example, via a gearbox. Doses of medication may be delivered to the user responsive to operation of a processor, in accordance with programming stored in memory associated with the processor or specifically when requested by the user, e.g., using a suitable wireless application on the user's smartphone. The processor may be configured to monitor one or more sensors and modify operation of pump 300 or alert the wearer based on information sensed by one or more sensors. Cap 400 is configured to receive medication from cartridge 500 and deliver predetermined doses of the medication through an outflow needle, into cannula 200, and to the wearer. Cap 400 preferably includes a microdosing system configured to measure and deliver the predetermined doses of medication.

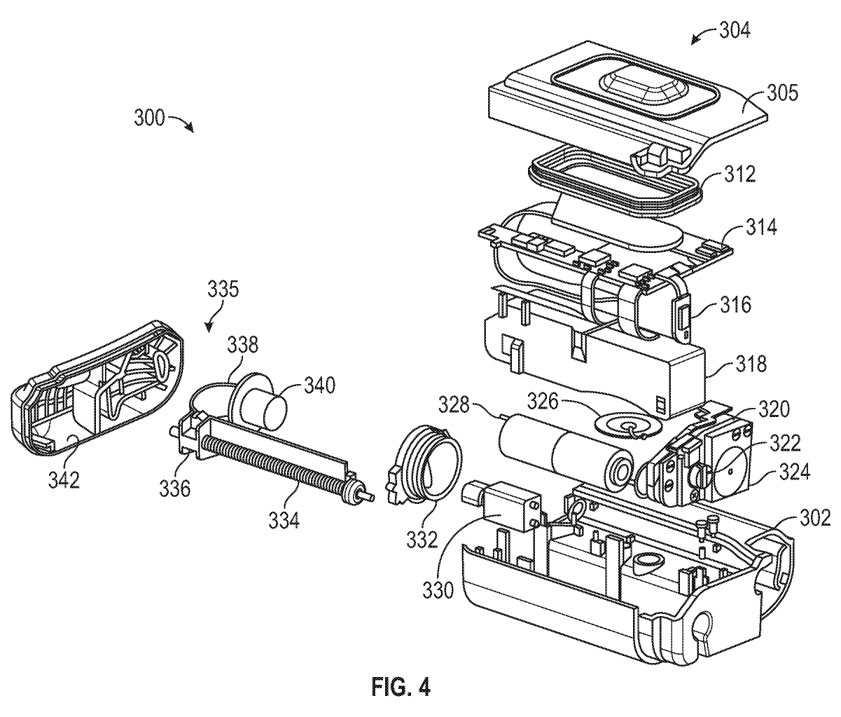

Referring now to FIG. 4, internal components of an exemplary pump are described. For example, pump 300 may include within pump housing 302, pump housing bottom 305 and pump housing back 342 the following components: coil 312, circuit board 314, sensor 316, battery 318, sensor 320, mechanical coupling 322, gearbox 324, sound generator 326, pump motor 328, vibration motor 330, cartridge holder 332, and/or pushing system 335 (which may include screw 334, nut 336, bendable rod 338, and/or pusher 340). Further details regarding these components can be found in U.S. Patent Publication No. 2023/0364332, which is hereby incorporated by reference herein in its entirety.

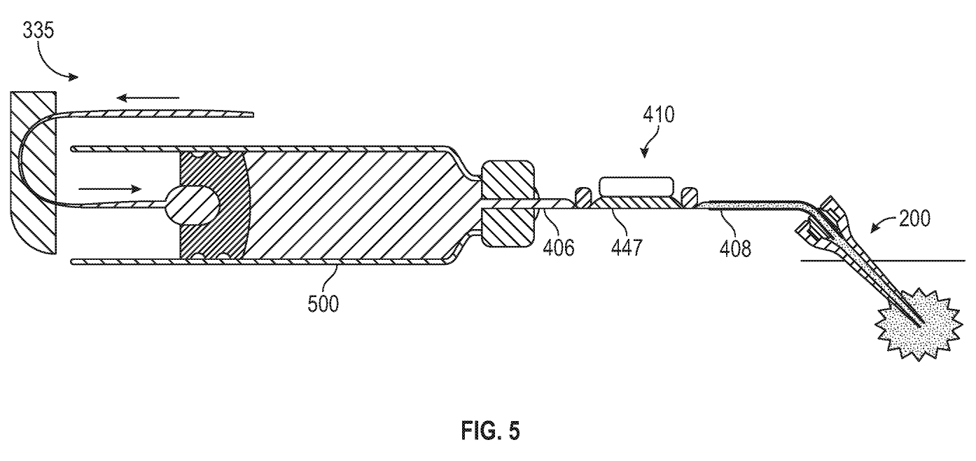

Referring now to FIG. 5, a schematic depiction of an exemplary pusher and microdosing system is described. Medication may be delivered from cartridge 500, through inflow needle 406, and into flattened dosing tube 447. Upon rotation of the microdosing system, the medication may then be forced out of flattened dosing tube 447, delivered through outflow needle 408 to cannula 200, and injected into the wearer. Preferably, microdosing system 410 and pushing system 335 work together to maintain the pressure within cartridge 500 within a predetermined pressure range. For example, the strain of the plunger and the bendable rod of pushing 335 within cartridge 500 at one end and the levers of microdosing system 410 at the other end create a closed system in which the medication is disposed, the closed system helping maintain the pressure within the predetermined pressure range. The motor within the pump simultaneously advances the plunger and activates microdosing system 410 at each dosing cycle. Preferably, the plunger advances in microsteps (e.g., 3-4 um, preferably 3.7 um) at each dosing cycle such that the pressure within cartridge 500 varies only minimally due to the “stick-slip” effect that occurs at the elastomeric portion of the plunger. The patch pump uses the constant pressure to refill the reservoir of flattened dosing tube 447 with equivalent volumes of medication at every dosing cycle. Inflow needle 406, outflow needle 408 and dosing tube 447 are preferably made from materials compatible with insulin. For example, the inflow and outflow needles may be made of stainless steel and dosing tube 447 may be made of fluoropolymer tubing. Because the flow path for insulin may be directly from cartridge 500 into inflow needle 406, then into dosing tube 447, then into outflow needle, then into cannula 200 (which is also made from insulin compatible material), all materials in contact with the insulin are insulin compatible.

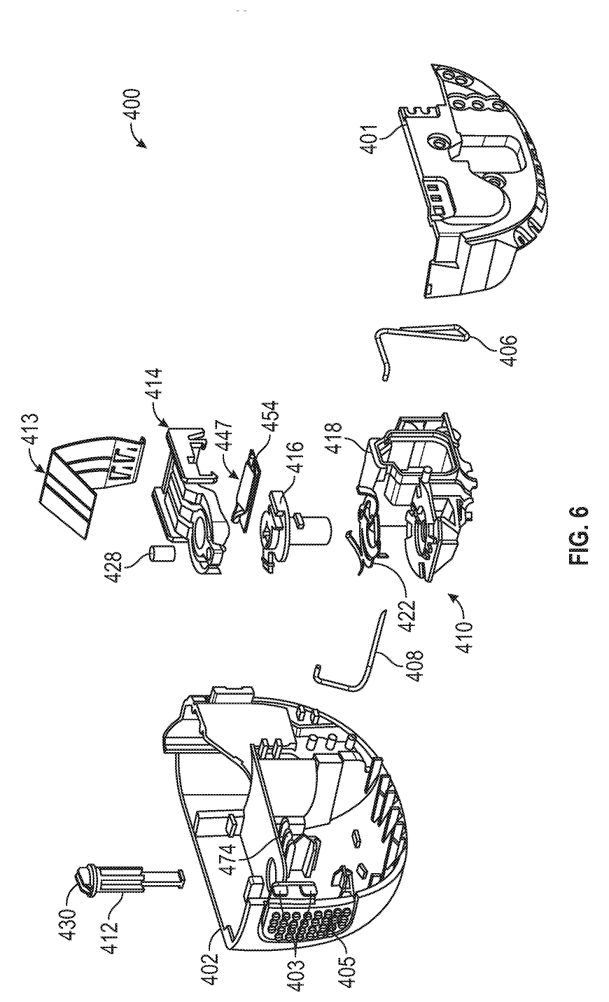

With respect to FIG. 6, an exploded view an exemplary cap is described. For example, cap 400 may include within cap housing 402 and internal cap housing 401 the following components; cap clips 403, unclipping buttons 405, inflow needle 406, outflow needle 408, microdosing system 410, cam shaft 412, lever spring system 413, lever system 414, camp late 416, microdosing structure 418, spring 422, magnet 428 and tabs 430, flattened dosing tube 447, dosing tube support 454, and/or prongs 474. Further details regarding these components can be found in U.S. Patent No. 11,529,460, which is hereby incorporated herein by reference.

The cap is configured to deliver medication from the cartridge to the wearer and preferably includes microdosing system 410 configured to measure and deliver the predetermined doses of medication. Preferably, microdosing system 410 is configured such that the insulin travels through a simple pathway designed for low shear stress, which avoids compromising the insulin. Microdosing system 410 may be configured to only deliver the predetermined dose of medication upon initialization of the microdosing system, when the pressure sensor, senses that the pressure within the cartridge is within the predetermined range. The initialization process helps ensure that the microdosing system accurately measures the predetermined doses of medication. The predetermined pressure range may depend upon the cartridge or medication used, but preferably is between 600 mbar and 1000 mbar. When the pressure sensor senses that the pressure is within the predetermined range, the processor, may be configured to execute programmed instructions stored in the memory to cause the microdosing system to move from an initialization position to a dosing position, such that medication may be delivered to the wearer.

Microdosing system 410 is configured to provide for more accurate dosing and to reduce the noise from the delivery of the medication. Microdosing system 410 preferably is coupled to an inflow needle, which may extend from the cartridge to microdosing system 410, and an outflow needle, which may extend from microdosing system 410 to the cannula. Coupled between the inflow and outflow needle is a dosing tube configured to receive the medication, the dosing tube having a flattened portion including a reservoir portion configured to hold the predetermined dose of medication. The reservoir portion may comprise one or more welded portions that help ensure that a predetermined volume of medication is delivered to the wearer.

Microdosing system 410 further may include a cam, which is configured to rotate, and lever system 414, which is configured to contact the dosing tube and release the predetermined dose of medication into the outflow needle upon interaction with the cam. The cam may be circular in shape to reduce the overall size of the cam and/or to permit two microdoses with a full 360 degree turn of the cam, although other shapes may be suitable. Lever system 414 may include one or more levers, each lever configured to be independently movable such that the movement of a first lever does not affect the position of a second lever. The cam may include cam shaft 412, which is oriented in a first plane, and cam plate 416, which is coupled to cam shaft 412 and oriented in a second plane, the second plane preferably orthogonal to the first plane. The cam plate may be circular in shape to reduce the overall size of the cam plate and/or to permit two microdoses with a full 360 degree turn of the cam plate, although other shapes may be suitable. Cam plate 416 may include a top surface having one or more raised surfaces that are configured to interact with one or more levers of lever system 414 upon rotation of cam shaft 412 such that the predetermined dose of medication is delivered to the wearer. The raised surface(s) may extend away from the circular portion of cam plate 416, such as in a direction generally parallel to the longitudinal axis of the cam. Lever system 414 further may include magnet 428, which is configured to be used to detect an occlusion in the dosing pathway.

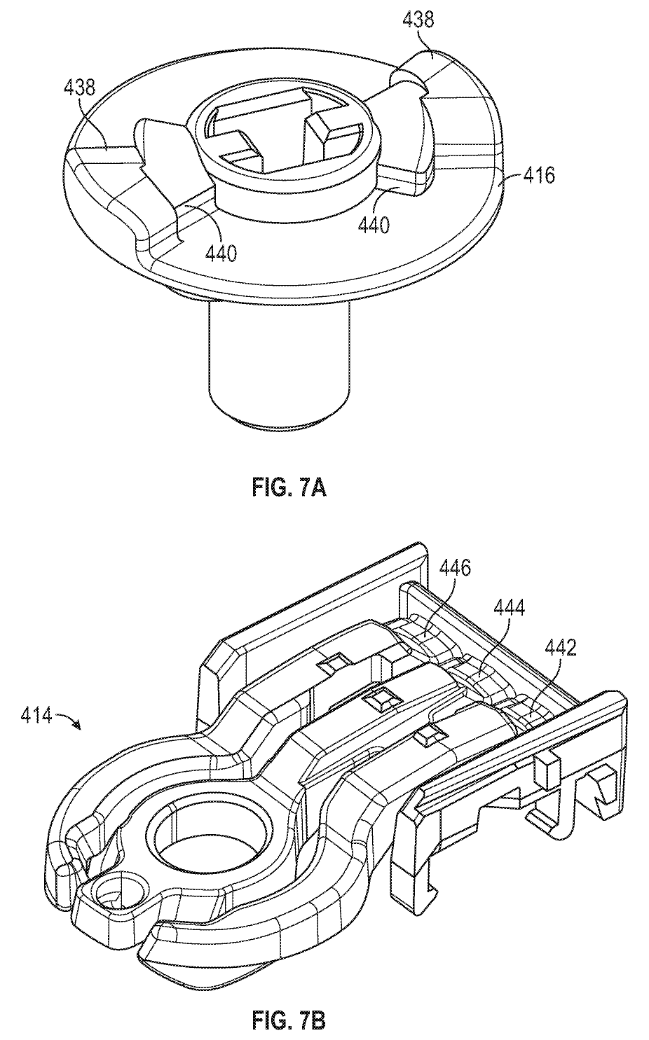

Referring now to FIGS. 7A and 7B, details of a circular cam and lever system are described. Cam plate 416 is configured to interact with lever system 414 such that, upon rotation of cam plate 416, the levers of lever system 414 move in a series of steps and deliver a predetermined dose of medication to the wearer. Cam plate 416 may include one or more rounded, raised surfaces that interact with corresponding rounded lever ramps on the levers of lever system 414. The rounded surfaces ensure smooth movement between of the levers and may help mitigate the sound of the microdosing system. Each time a lever contacts the raised surfaces of cam plate 416, the lever transitions from a lowered position to a raised position, allowing medication to flow through the corresponding section of the dosing tube.

Cam plate 416 may include outer raised surfaces 438 and inner raised surfaces 440, positioned radially inward of outer raised surfaces 438. Outer raised surfaces 438 may be configured to contact the first lever ramp of first lever 442 and the second lever ramp of second lever 446. Preferably, outer raised surfaces 438 are sized and shaped such that the outer raised surface may be disposed between the first lever ramp and the second lever ramp without contacting either the first lever ramp or the second lever ramp. Inner raised surfaces 440 may be configured to contact only the middle lever ramp of middle lever 444. The raised surfaces on cam plate 416 may be configured such that a complete 360-degree rotation of cam plate 416 delivers two predetermined doses of medication towards the wearer. For example, in FIG. 7A, cam plate 416 includes two outer raised surfaces 438 and two inner raised surfaces 440, the second outer and inner raised surfaces mirror images of the first outer and inner raised surfaces. As will be understood by one of ordinary skill in the art, cam plate 416 may include more than two raised surfaces and may be configured such that a 360-degree rotation or a rotation of less than 180 degrees is required for delivery of a predetermined dose of medication.

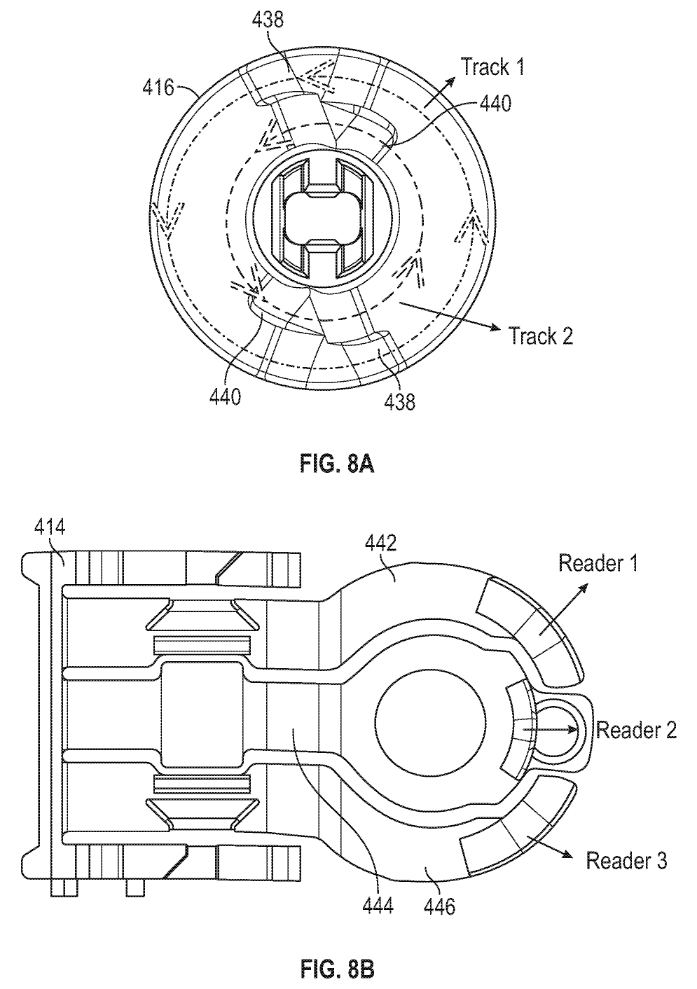

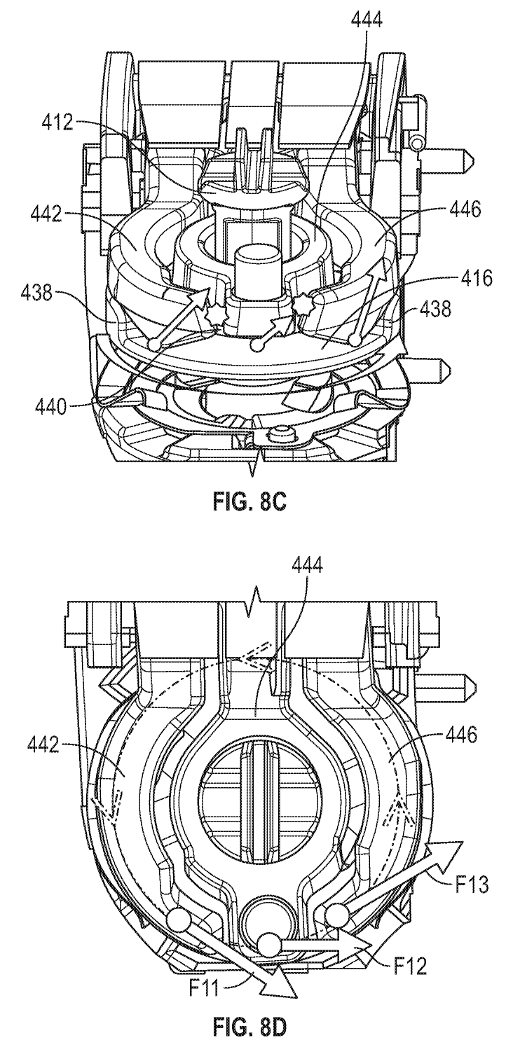

However, it has been found that due to the flexibility of the levers, during these operations the levers can be laterally deflected and pushed against each other affecting the operation of the levers. In particular, referring to FIGS. 8A-8B, as noted above the cam plate 416 includes outer raised surfaces 438 defining a first, outer track (Track 1) and inner raised surfaces 440 defining a second, outer track (Track 2). Reader 1 and Reader 3 use the outer Track 1 and Reader 2 uses the inner Track 2. Referring now to FIGS. 8C-8D, when the cam plate 416 is rotating (counterclockwise), the levers 442, 444 and 446 are lifted when coming into contact with the corresponding raised surfaces 438, 440 as is necessary in order for the levers to interact with the dosing tube to dispense medicament. However, the flexibility required for the levers to be raised and lowered can cause the levers to also be deflected sideways into each other, creating a parasitic effect in which each lever affects proper operation of the adjacent levers. In particular, as can be seen in FIGS. 8C-8D, the first lever 442 can be pushed against the second lever 444 to impair the function of the second lever 444, the second lever 444 can be pushed against the third lever 446 impairing the function of the third lever 446 and the third lever 446 can be pushed off of the track to contact other parts within the pump, impairing the functioning of the third lever 446.

As can be seen from the above, only vertical movement of the levers is desired. Embodiments disclosed herein therefore provide a system that minimizes the horizontal, parasitic movement of levers.

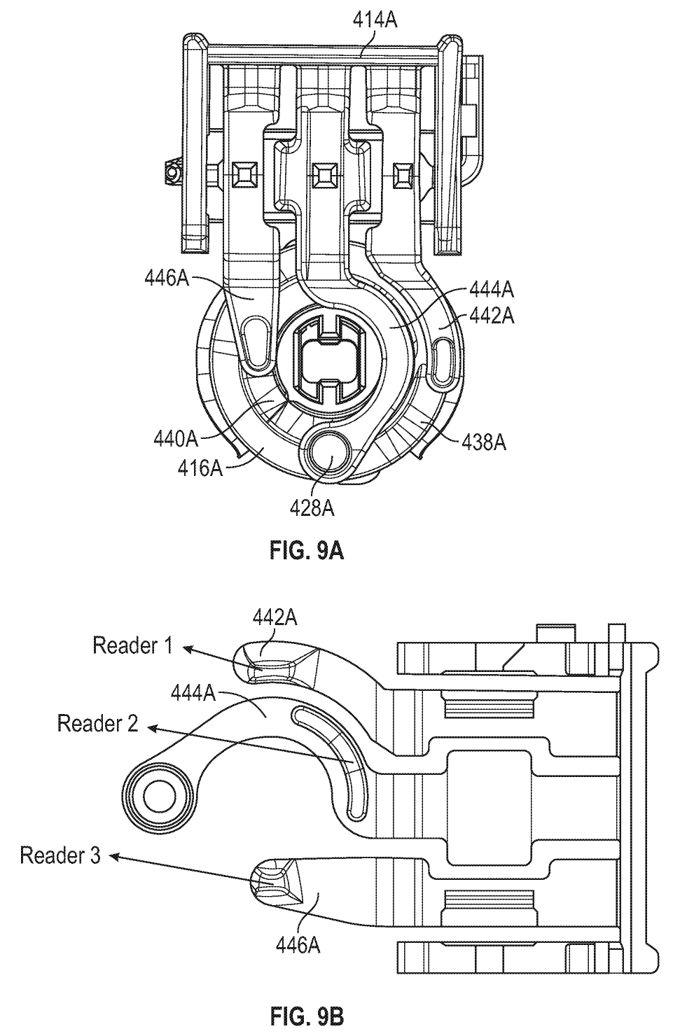

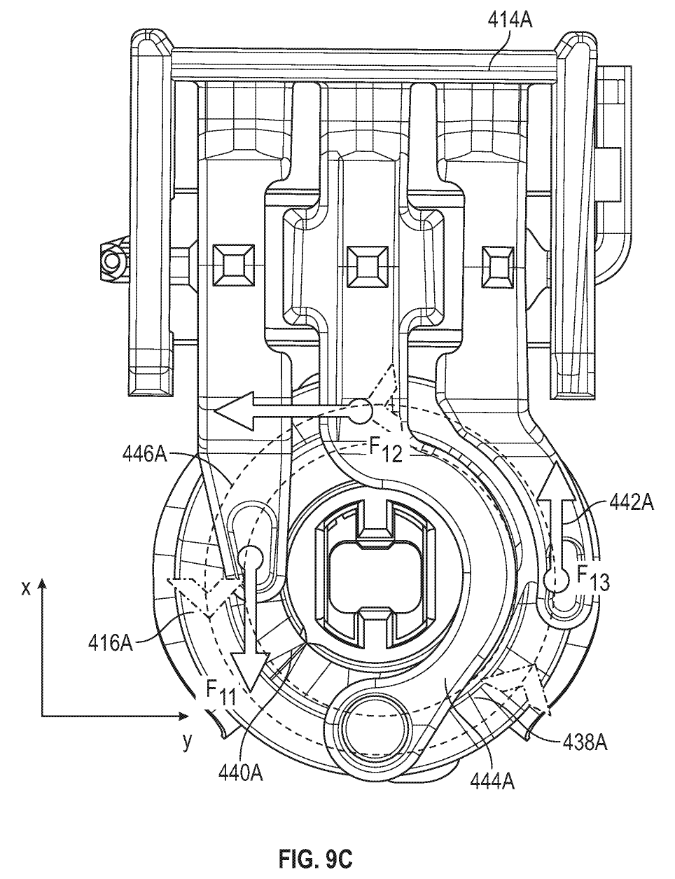

In particular, FIGS. 9A-9C depict a lever system 414A and cam plate 416A according to the present disclosure that addresses these issues. Lever arms 442A, 444A, 446A on lever system 414A and outer 438A and inner 440A raised surfaces on cam plate 416A have been provided with different configurations such that the force vector on the lever arms from the raised surfaces minimizes the parasitic lateral movement of lever arms described above.

This result is primarily generated by two techniques. First, the contact points where Reader 1 and Reader 3 on first lever 442A and third lever arm 444A contact the outer raised surfaces 438A, respectively, are positioned such that the force vector (see vectors F₁₁ and F₁₃ in FIG. 9C) on each puts the corresponding reader primarily in compression or extension (x-axis), which minimizes flexion in the y-direction (i.e., lateral displacement). The contact point on Reader 2 on lever arm 444A was moved closer to the lever fixation point. This significantly reduces any horizontal deflection from force vector F₁₂ in FIG. 9C compared to the deflection that would be generated from such a force vector at a more distal point of the lever arm 444A. In this manner, as the cam plate 416A rotates the lever arms of the lever system 414A are constrained to moving between raised and lowered positions without deflecting laterally and interfering with the functioning of the adjacent levers.

As with the previous embodiment, lever system 414A including lever arms may be unitarily formed as a single monolith construct with the lever arms cantilevered from a common fixation point/arm. However, the overall configurations of the lever arms themselves are =different in FIGS. 9A-9C from those in FIGS. 8A-8D. For example, in FIGS. 8A-8D, the three lever arms 442, 444, 446 extend the same length from the fixation point. In contrast, lever arms 442A, 442B, 442C in FIGS. 9A-9C have differing lengths. In particular, the middle lever arm 444A extends significantly further out from the fixation point/arm of the lever than the other two lever arms. This lever arm 444A does so because it accommodates a magnet 428A that is positioned over the cam plate 416 used to sense occlusions or other system conditions as described in the applications incorporated by reference herein. Lever arm 442A and lever arm 446A extend shorter distances from their fixation points as they need only extend as far as necessary to properly position the relocated Readers 1, 3 disposed therein (whereas, as shown in FIG. 9B, lever arm 444A extends significantly beyond Reader 2 in order to position the magnet 428A on the far end of cam plate 416A). This reduces the torque on these lever arms when contacting the cam plate 416A, which further contributes to a reduction in lateral deflection of the arms and potential interference with adjacent levers. In addition, lever arm 442A and lever arm 446A may extend different lengths from their corresponding fixation points relative to each other. One reason this is possible is because these lever arms interface with different tracks at different locations on the cam plate (lever arm 442A interfaces with Track 1 on the outer edge of cam plate and lever arm 446 with Track 2 on the inner portion of cam plate). In the depicted embodiment, however, lever arm 442A extends slightly further than lever arm 446A (despite the outer Track 1 being closer). This is due to the need for the middle lever arm 444A to curve around the central opening in the cam plate (which accommodates cam shaft 412) and in this embodiment the arm curves towards lever arm 442A necessitating a curve in that lever arm, whereas lever arm 446A can extend more directly linearly onto cam plate.

In embodiments, a medication infusion device comprises a patch pump configured to be removably adhered to a wearer’s skin for delivering doses of medication from a container through a transcutaneous portion. The patch pump can include a dosing tube configured to receive medication, a plurality of levers configured to contact the dosing tube to move a predetermined dose of medication towards the transcutaneous portion and a circular cam comprising a shaft oriented in a first plane and a circular plate oriented in a second plane. The circular plate can be coupled to the shaft and include a top surface having one or more raised surfaces extending from the top surface configured to move each of the plurality of levers from a lowered position to a raised position in a series of steps upon rotation of the shaft. Contact surfaces on the plurality of levers at which the plurality of levers are contacted by the raised surfaces can be configured to inhibit lateral movement of each the plurality of levers when the plurality of levers are moved from the lowered position to the raised position. Rotation of the shaft can deliver the predetermined dose of medication towards the wearer.

In some embodiments, the plurality of levers are cantilevered from a common fixation arm.

In some embodiments, the plurality of levers and the common fixation arm are unitarily formed as a single monolithic construct.

In some embodiments, each of the plurality of levers terminates at a different distance from the common fixation arm.

In some embodiments, the contact surface on at least one of the plurality of levers is a different distance from the common fixation arm than the contact surface on at least one other of the plurality of levers.

In some embodiments, the device further includes a magnet disposed on one of the plurality of levers, the magnet being disposed over the circular plate of the cam.

In some embodiments, each of the plurality of levers has a different shape.

In some embodiments, the one or more raised surfaces includes at least one raised surface on a first track and at least one raised surface on a second track on the top surface of the circular plate, the first track and the second track being located at different radial locations on the top surface.

In some embodiments, the contact surface of at least one of the plurality of levers is disposed along the first track and the contact surface of at least one of the plurality of levers is disposed along the second track.

In some embodiments, a force on one or more of the plurality of levers from the one or more raised surfaces of the circular cam contacting the contact surface of the one or more of the plurality levers is in a direction along a length of the one or more of the plurality of levers.

In embodiments, a medication infusion device comprises a patch pump configured to be removably adhered to a wearer’s skin for delivering doses of medication from a container through a transcutaneous portion. The patch pump can include a dosing tube configured to receive medication, a plurality of levers configured to contact the dosing tube to move a predetermined dose of medication towards the transcutaneous portion and a circular cam comprising a shaft oriented in a first plane and a circular plate oriented in a second plane. The circular plate can be coupled to the shaft and include a top surface having one or more raised surfaces extending from the top surface configured to move each of the plurality of levers from a lowered position to a raised position in a series of steps upon rotation of the shaft. Contact surfaces on the plurality of levers at which the plurality of levers are contacted by the raised surfaces can be positioned such that forces transmitted to the plurality of levers by the raised surfaces when the plurality of levers are moved from the lowered position to the raised inhibit lateral movement of each the plurality of levers. Rotation of the shaft can deliver the predetermined dose of medication towards the wearer.

In some embodiments, the plurality of levers are cantilevered from a common fixation arm.

In some embodiments, the plurality of levers and the common fixation arm are unitarily formed as a single monolithic construct.

In some embodiments, each of the plurality of levers terminates at a different distance from the common fixation arm.

In some embodiments, the contact surface on at least one of the plurality of levers is a different distance from the common fixation arm than the contact surface on at least one other of the plurality of levers.

In some embodiments, the device can further include a magnet disposed on one of the plurality of levers, the magnet being disposed over the circular plate of the cam.

In some embodiments, each of the plurality of levers has a different shape.

In some embodiments, the one or more raised surfaces includes at least one raised surface on a first track and at least one raised surface on a second track on the top surface of the circular plate, the first track and the second track being located at different radial locations on the top surface.

In some embodiments, the contact surface of at least one of the plurality of levers is disposed along the first track and the contact surface of at least one of the plurality of levers is disposed along the second track.

In some embodiments, a force on one or more of the plurality of levers from the one or more raised surfaces of the circular cam contacting the contact surface of the one or more of the plurality levers is in a direction along a length of the one or more of the plurality of levers.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to producenumerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

1. A medication infusion device comprising a patch pump configured to be removably adhered to a wearer’s skin for delivering doses of medication from a container through a transcutaneous portion, the patch pump comprising:
a dosing tube configured to receive medication;
a plurality of levers configured to contact the dosing tube to move a predetermined dose of medication towards the transcutaneous portion;
a circular cam comprising a shaft oriented in a first plane and a circular plate oriented in a second plane, the circular plate coupled to the shaft and comprising a top surface having one or more raised surfaces extending from the top surface, the one or more raised surfaces configured to move each of the plurality of levers from a lowered position to a raised position in a series of steps upon rotation of the shaft,
wherein contact surfaces on the plurality of levers at which the plurality of levers are contacted by the raised surfaces are configured to inhibit lateral movement of each the plurality of levers when the plurality of levers are moved from the lowered position to the raised position,
wherein rotation of the shaft delivers the predetermined dose of medication towards the wearer.

2. The medication infusion device of claim 1, wherein the plurality of levers are cantilevered from a common fixation arm.

3. The medication infusion device of claim 2, wherein the plurality of levers and the common fixation arm are unitarily formed as a single monolithic construct.

4. The medication infusion device of claim 2, wherein each of the plurality of levers terminates at a different distance from the common fixation arm.

5. The medication infusion device of claim 2, wherein the contact surface on at least one of the plurality of levers is a different distance from the common fixation arm than the contact surface on at least one other of the plurality of levers.

6. The medication infusion device of claim 1, further comprising a magnet disposed on one of the plurality of levers, the magnet being disposed over the circular plate of the cam.

7. The medication infusion device of claim 1, wherein each of the plurality of levers has a different shape.

8. The medication infusion device of claim 1, wherein the one or more raised surfaces includes at least one raised surface on a first track and at least one raised surface on a second track on the top surface of the circular plate, the first track and the second track being located at different radial locations on the top surface.

9. The medication infusion device of claim 8, wherein the contact surface of at least one of the plurality of levers is disposed along the first track and the contact surface of at least one of the plurality of levers is disposed along the second track.

10. The medication infusion device of claim 1, wherein a force on one or more of the plurality of levers from the one or more raised surfaces of the circular cam contacting the contact surface of the one or more of the plurality levers is in a direction along a length of the one or more of the plurality of levers.

11. A medication infusion device comprising a patch pump configured to be removably adhered to a wearer’s skin for delivering doses of medication from a container through a transcutaneous portion, the patch pump comprising:
a dosing tube configured to receive medication;
a plurality of levers configured to contact the dosing tube to move a predetermined dose of medication towards the transcutaneous portion;
a circular cam comprising a shaft oriented in a first plane and a circular plate oriented in a second plane, the circular plate coupled to the shaft and comprising a top surface having one or more raised surfaces extending from the top surface, the one or more raised surfaces configured to move each of the plurality of levers from a lowered position to a raised position in a series of steps upon rotation of the shaft,
wherein contact surfaces on the plurality of levers at which the plurality of levers are contacted by the raised surfaces are positioned such that forces transmitted to the plurality of levers by the raised surfaces when the plurality of levers are moved from the lowered position to the raised inhibit lateral movement of each the plurality of levers,
wherein rotation of the shaft delivers the predetermined dose of medication towards the wearer.

12. The medication infusion device of claim 11, wherein the plurality of levers are cantilevered from a common fixation arm.

13. The medication infusion device of claim 12, wherein the plurality of levers and the common fixation arm are unitarily formed as a single monolithic construct.

14. The medication infusion device of claim 12, wherein each of the plurality of levers terminates at a different distance from the common fixation arm.

15. The medication infusion device of claim 12, wherein the contact surface on at least one of the plurality of levers is a different distance from the common fixation arm than the contact surface on at least one other of the plurality of levers.

16. The medication infusion device of claim 11, further comprising a magnet disposed on one of the plurality of levers, the magnet being disposed over the circular plate of the cam.

17. The medication infusion device of claim 11, wherein each of the plurality of levers has a different shape.

18. The medication infusion device of claim 11, wherein the one or more raised surfaces includes at least one raised surface on a first track and at least one raised surface on a second track on the top surface of the circular plate, the first track and the second track being located at different radial locations on the top surface.

19. The medication infusion device of claim 18, wherein the contact surface of at least one of the plurality of levers is disposed along the first track and the contact surface of at least one of the plurality of levers is disposed along the second track.

20. The medication infusion device of claim 11, wherein a force on one or more of the plurality of levers from the one or more raised surfaces of the circular cam contacting the contact surface of the one or more of the plurality levers is in a direction along a length of the one or more of the plurality of levers.
